(19) United States
(12) Patent Application Publication
(10) Pub. No.: US 2012/0080350 A1
(43) Pub. Date: Apr. 5, 2012

(54) MEDICATION STORAGE LOCK BOX HAVING A SEVERABLE STRAP COUPLING THE LID TO A LOCKING CLASP

(76) Inventor: Michael Mac Powell, Lehi, UT (US)

(21) Appl. No.: 12/897,780

(22) Filed: Oct. 4, 2010

(51) Int. Cl. A61B 19/02

(52) U.S. Cl. 206/528; 220/265

(57) ABSTRACT

A medication storage lock box that can be easily opened in an emergency includes a storage chamber to which a lid is hingeably connected. A severable strap couples the lid to a clasp that engages the locking mechanism of a combination lock. The lid may be opened either by entering the proper lock combination and releasing the clasp or, in a medical emergency caused by the owner becoming incapacitated or forgetting the combination of the lock, by cutting the strap which couples the lid to the clasp. The lock box may be made of materials such as leather, imitation leather, fiberboard-reinforced fabric, fiberboard, thermoplastic resins, thermosetting resins, or resin impregnated paper.₍₁₁₂₎

This application has a priority data based on the filing of Provisional Patent Application No. 61/248,199 on Oct. 2, 2009 titled MEDICATION STORAGE LOCK BOX HAVING A SEVERABLE STRAP COUPLING THE LID TO A LOCK CLASP.

### FIELD OF THE INVENTION

This invention relates, generally, to lock boxes and, more particularly, to lock boxes designed specifically for the safe storage of prescription medications.

### BACKGROUND OF THE INVENTION

The unauthorized use of prescription pain medications has reached epidemic proportions. The list of opiate-derived pain medications is extensive, and includes Dilaudid (hydromorphone, an opiate narcotic), Lortab (a combination of the narcotic hydrocodone and acetaminophen), Darvocet-N 100 (a combination of Propoxyphene—a narcotic and acetaminophen), Dilaudid (hydomorphone), MS Contin (oxycodone, a opiate narcotic), OxyContin (an opiate narcotic), Percocet (a combination of oxycodone and acetaminophen), Tylenol with Codeine (an opiate narcotic), Ultracet (a combination of tramadol, an opiate narcotic, and acetaminophen, Ultram (a narcotic), and Vicodin (a combination of hydrocodone an acetaminophen). The list of non-narcotic, mind-altering drugs is also extensive and includes drugs such as Valium (diazepam), Xanax (alprazolam) and countless others.

A major problem encountered in our society is that by prescribing addictive and mind-altering drugs to patients who ostensibly need them, those with whom they live may be exposed to those drugs out of curiosity or even by accident. A number of lockable medication cabinets and medication lock boxes are widely available in the marketplace. One such medication storage lock box, which bears the trademark LOCK MED®, appears to be fabricated of tough stainless steel, and is equipped with a triple-disc combination lock having 1000 different permutations. Typically, these lockable devices are quite resistant to tampering, and forcing or breaking them open would require access to one or more special tools, such as a grinder, a pry bar, a metal saw, and hammer. Because the majority of prescription medications are taken by elderly patients, there is reasonable possibility that a patient may experience a dangerous medical condition if the medications are not taken on a regular schedule. Interference with the schedule may occur under at least two scenarios. The first is that the patient may forget the combination. Ongoing anxiety related to the initial occurrence of forgetfulness may prevent the patient from remembering the combination for an extended period, which may greatly exceed the recommended gap between administrations of the medication. A second scenario is that the patient may become incapacitated, which may prevent him from accessing the locked medications on his own.

What is needed is a relatively inexpensive medication storage lock box that can be easily opened in an emergency. However, the emergency opening of the lock box will result in the obvious destruction of the secure storage capability of the box. Consequently, the box will need to be replaced in order to regain secure storage capability.

### SUMMARY OF THE INVENTION

The present invention provides an apparatus that fulfills the heretofore expressed need for an apparatus for an inexpensive medication storage lock box that can be easily opened in an emergency. The lock box has a storage chamber to which a lid is hingeably connected. A short strap couples the lid to a clasp that engages the locking mechanism of a combination lock. The lid may be opened either by entering the proper lock combination and releasing the clasp, or by cutting the strap which couples the lid to the clasp. Once the strap is cut, the lock box is no longer secure, and must be replaced. The reasoning behind the use of such a device is that there is no such a thing as a lock box that cannot be opened by a determined individual. Furthermore, if the strap is cut by an unauthorized individual, the patient-owner of the medications will be on notice that his medications may have been stolen, and police may be contacted if the cutting of the strap involves a theft of narcotics. In any case, the owner of the lock box is protected against both forgetting the combination of the combination lock and incapacity where he is unable to open the lock box himself. In either instance, the lock box can be easily opened with a sharp knife or pair of scissors. Given that the box is readily, but identifiably breachable, there is no need to utilize safe-like fabrication techniques. To the contrary, the lock box may be made of materials such as leather, imitation leather, fiberboard-reinforced fabric, fiberboard, thermoplastic resins, thermosetting resins, or resin impregnated paper.

### BRIEF DESCRIPTION OF THE DRAWINGS

FIG. 1 is an isometric view of a closed medication storage lock box having a severable strap which secures the top of the lock box to the locking latch of the combination lock;

FIG. 2 is an isometric view of the medication storage lock box of FIG. 1 following its opening with the lockable latch to expose the contents of the lock box; and

FIG. 3 is an isometric view of the medication storage lock box of FIG. 1 following the cutting of the severable strap and opening of the top to expose the contents of the lock box.

### PREFERRED EMBODIMENT OF THE INVENTION

The invention will now be described in detail with reference to the attached drawing figures. It is to be understood that the drawings are not necessarily drawn to scale and that they are intended to be merely illustrative. Although a generally rectangular lock box is shown and described, it should be obvious that it is only one of many possible shapes, and that the feature of a severable strap coupling the lid to the latch of the combination lock may be readily adapted to a plethora of lock boxes having different shapes.

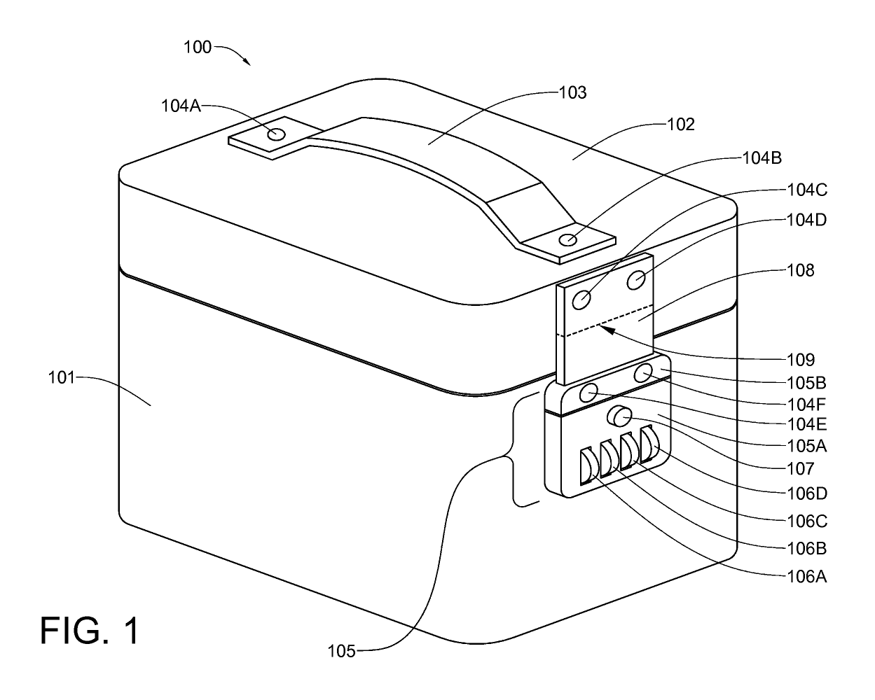

Referring now to FIGS. 1, 2 and 3, a medication storage lock box 100 incorporating the essential features of the present invention is shown in a closed configuration. The lock box 100 includes a lower chamber portion 101 and a lid portion 102. A handle 103 is secured to an upper major face of the lid portion 102 with rivets 104A and 104B. The lid portion 102 can be secured to the lower chamber portion 101 with a combination lockset 105. A bottom portion of the combination lockset 105A is secured to the lower chamber portion 101. A severable strap 108 is secured to the lid portion 102 with rivets 104C and 104D and to an upper portion 105B of the combination lockset 105 with rivets 104E and 104F. The use of rivets is an additional security feature, as the severable strap 108 cannot easily be surreptitiously disconnected and reconnected with, for example, a releasable adhesive. The upper portion of the combination lockset 105B is equipped with a latch 202, which engages the bottom portion of the combination lockset 105B through an aperture 203. The combination lockset is equipped with four rotatable combination wheels 106A, 106B, 106C and 106D. When the four wheels are set on a proper combination, the latch is disengaged from the lower portion 105A of the combination lockset 105. A lower rear edge of the lid portion 102 is hingeably connected to the lower chamber portion 101 along a upper rear edge thereof. The hinge can be a live hinge with case material coupling the lid portion 102 to the lower chamber portion 101. Alternatively, hinges such as those used on small rigid suitcases and rigid brief cases, can be riveted or bonded to both the lid 105 and to the lower chamber portion 101.

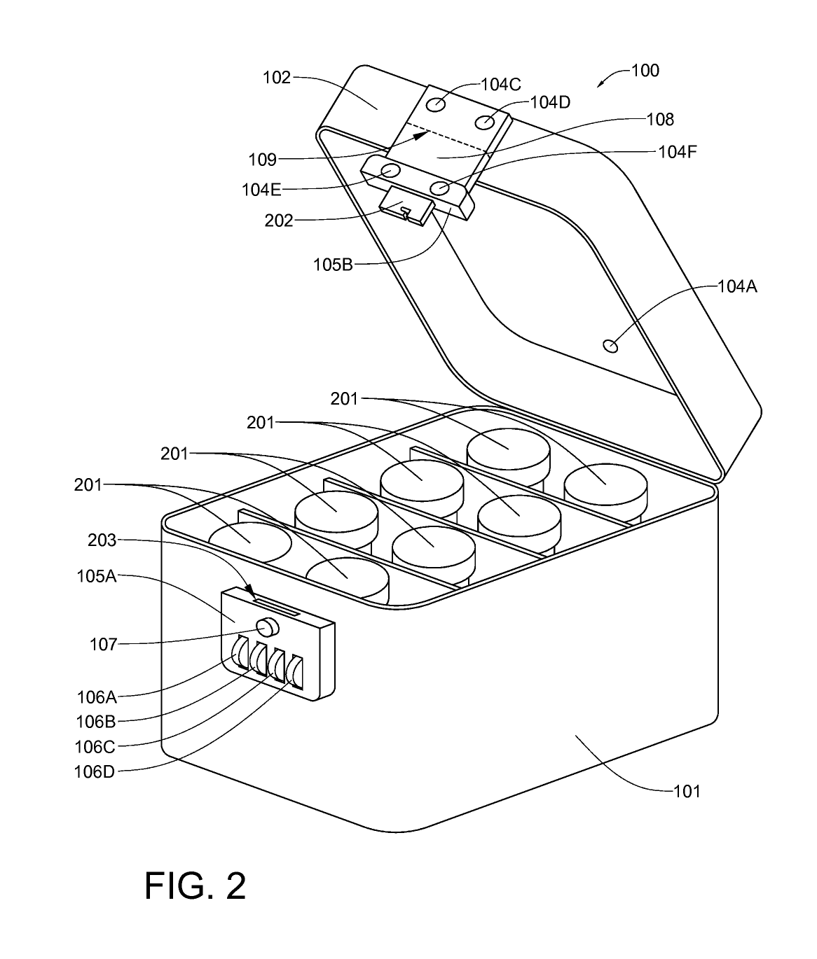

Referring now, especially to FIG. 2, the lock box 100 has been opened using the proper combination of the combination lockset 105. Eight prescription bottles 201 have been placed within the lower chamber portion 101. IN this case, the lock box 100 is shown having a live hinge with case material coupling the lid portion 102 to the lower chamber portion 101.

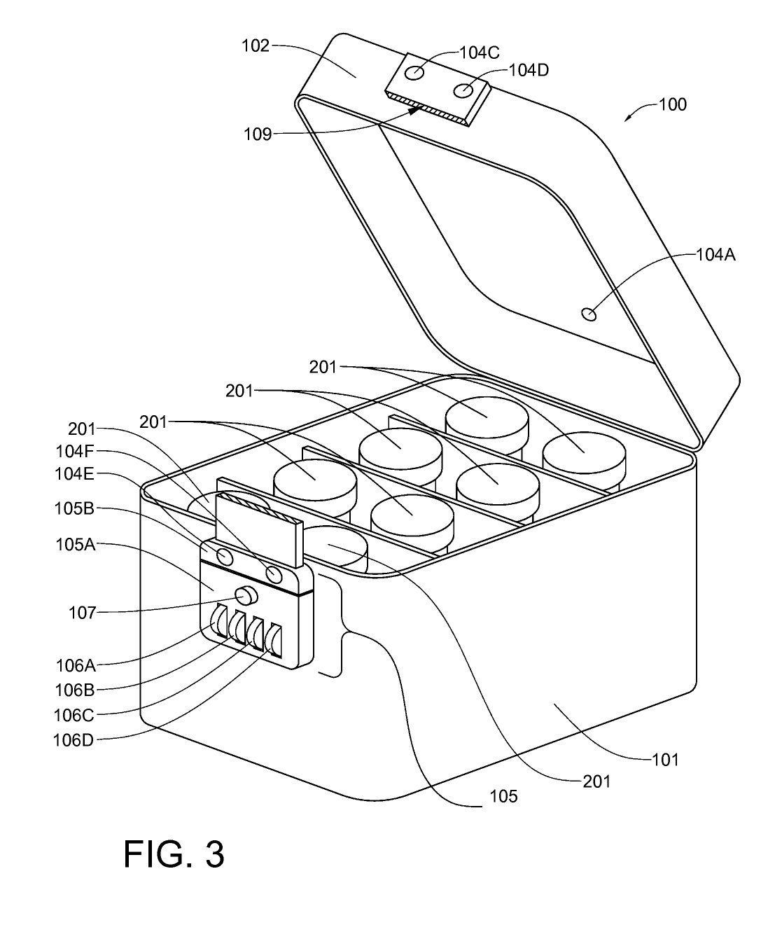

Referring now, especially, to FIG. 3, the severable strap 108 of the medication storage lock box 100 has been cut in the middle thereof at cut line 109. The severable strap can be cut if the owner forgets the combination or if a care giver must provide medication during an emergency while the patient owner of the lock box 100 is incapacitated and unable to open it himself. However, the lock box 100 is designed so that the box cannot be opened surreptitiously by cutting the severable strap 108, thereby alerting the owner if others have breached the security of the lock box. Once the severable strap 108 has been cut, the medication storage lock box 100 can no longer provide secure storage unless it is professionally repaired, which may be more costly than simply purchasing another similar lock box.

The lid 102 and the lower chamber portion 101 of the lock box 100 may be made of materials such as leather, imitation leather, fiberboard-reinforced fabric, fiberboard, thermoplastic resins, thermosetting resins, or resin impregnated paper. The severable strap may be made of leather, webbing, a tough multi-layer flexible fabric, a tough thermoplastic polymer, such as polypropylene, fabric-reinforced rubber, or even a soft metal such as copper.

Although only a single embodiment of the medication storage lock box has been shown and described, it will be obvious to those having ordinary skill in the art that changes and modifications may be made thereto without departing from the scope and the spirit of the invention as hereinafter claimed.

1. A medication storage lock box comprising:
a lower chamber portion;
an upper lid portion hingedly coupled to the lower chamber portion; and
a severable strap, an end thereof being permanently secured to the medication storage lock box, said severable strap coupling the lower chamber portion to the upper lid portion via a lockable clasp.

2. The medication storage lock box of claim 1, wherein said lower chamber portion and said upper lid portion are made of materials selected from the group consisting of leather, imitation leather, fiberboard-reinforced fabric, fiberboard, thermoplastic resins, thermosetting resins, and resin impregnated paper.

3. The medication storage lock box of claim 1, wherein said lockable clasp employs a combination lock.

4. The medication storage lock box of claim 1, wherein said severable strap is fabricated from a material selected from the group consisting of leather, webbing, tough multi-layer flexible fabric, tough thermoplastic polymers, fabric-reinforced rubber and soft metals.

5. A medication storage lock box comprising:
a lower chamber portion;
an upper lid portion hingedly coupled to the lower chamber portion;
a combination lock secured to the lower chamber portion;
a clasp which is lockably securable to the combination lock; and
a severable strap positioned on a single side of said medication storage lock box, said severable strap having a first end permanently secured to the upper lid portion and a second end secured to the clasp.

6. The medication storage lock box of claim 5, wherein said lower chamber portion and said upper lid portion are made of materials selected from the group consisting of leather, imitation leather, fiberboard-reinforced fabric, fiberboard, thermoplastic resins, thermosetting resins, and resin impregnated paper.

7. The medication storage lock box of claim 5, wherein said severable strap is fabricated from a material selected from the group consisting of leather, webbing, tough multi-layer flexible fabric, tough thermoplastic polymers, fabric-reinforced rubber and soft metals.

8. A medication storage lock box comprising:
a lower chamber portion;
an upper lid portion hingedly coupled to the lower chamber portion;
a combination lock;
a clasp securable to said lower chamber portion with said combination lock; and
a severable strap having a first end permanently secured to the upper lid portion and a second end secured to said clasp, said severable strap being positioned on a single side of said medication storage lock box.

9. The medication storage lock box of claim 8, wherein said lower chamber portion and said upper lid portion are made of materials selected from the group consisting of leather, imitation leather, fiberboard-reinforced fabric, fiberboard, thermoplastic resins, thermosetting resins, and resin impregnated paper.

10. The medication storage lock box of claim 8, wherein said severable strap is fabricated from a material selected from the group consisting of leather, webbing, tough multi-layer flexible fabric, tough thermoplastic polymers, fabric-reinforced rubber and soft metals.

11. The medication storage lock box of claim 1, wherein said end is permanently secured to said medication storage lock box with rivets.

12. The medication storage lock box of claim 5, wherein said first end is riveted to said upper lid portion.

13. The medication storage lock box of claim 8, wherein said first end is riveted to said upper lid portion.
